(19) United States
(12) Patent Application Publication
(10) Pub. No.: US 2006/0270060 A1
(43) Pub. Date: Nov. 30, 2006

(54) Rapid test for glycated albumin in saliva

(76) Inventor: Henry John Smith, Temecula, CA (US)

Correspondence Address: HENRY J. SMITH, 40670 CALLE TORCIDA, TEMECULA, CA, 92591, US

(21) Appl. No.: 11/140,306

(22) Filed: May 31, 2005

(51) Int. Cl. G01N 33/558; C12M 1/34

(52) U.S. Cl. 436/514; 435/287.2

(57) ABSTRACT

This invention describes a rapid immunochromatographic assay for measuring the ratio of glycated albumin to total albumin in saliva. Patients with diabetes have elevated levels of glucose in their blood that can react with plasma albumin to form glycated albumin. The amount of glycated albumin formed is directly correlated with the level of plasma glucose that the albumin has been exposed to over a period of time. Saliva albumin is derived from plasma albumin and therefore contains glycated and non-glycated albumin fractions that can be measured. The ratio of glycated albumin to total albumin in saliva will provide an indication of the average amount of protein glycation that occurred over the preceding 2-3 week period. The test is performed using a disposable strip that contains the testing reagents and the results are measured in a measuring instrument that automatically reads, calculates and displays the final result. The results of tests performed over a period of time are stored in the instrument's memory and presented in a numerical or graphical format so that the individual's glycated albumin level can be monitored over time.

### CROSS-REFERENCE TO RELATED APPLICATIONS

None

### FEDERALLY SPONSORED RESEARCH OR DEVELOPMENT

None

### BACKGROUND OF THE INVENTION

Diabetes mellitus or diabetes is a disease characterized by elevated levels of plasma glucose. Uncontrolled hyperglycemia is associated with increased risk of vascular disease including, nephropathy, neuropathy, retinopathy, hypertension, and death. There are two major forms of diabetes. Type 1 diabetes (or insulin-dependent diabetes) and Type 2 diabetes (or noninsulin-dependent diabetes). The American Diabetes Association has estimated that approximately 6% of the world population has diabetes.

The goal of diabetic therapy is to maintain a normal level of glucose in the blood. The American Diabetic Association has recommended that diabetics monitor their blood glucose level at least three times a day in order to adjust their insulin dosages and/or their eating habits and exercise regimen. However, glucose tests can only measure a point in time result and does not provide an overall assessment of glycemic control over a period of time.

To assess glycemic control over an extended period of time it is also recommended that hemoglobin A1c (glycated hemoglobin) testing be done 2-4 times a year. When blood proteins including hemoglobin are exposed to glucose over a period of time they become glycosylated and the degree of glycosylation is dependent on the average concentration of glucose and the length of time the proteins were exposed to the glucose. The level of glycated hemoglobin is also dependent upon the half-life of the hemoglobin molecule within the body. The net result is that measurement of glycated hemoglobin provides an estimate of the degree of glycosylation that occurred over the preceding 2-3 months.

It would be desirable to have a test that would provide an earlier indication of glycemic control to allow earlier therapeutic intervention. It would also be desirable to have a test that did not require the invasive process of obtaining a blood sample.

It would also be diserable to develop a simplified point-of-care assay that could be utilized in a doctor's office or by the patient at home.

### BRIEF SUMMARY OF THE INVENTION

This invention describes a non-invasive method of measuring glycated albumin compared to total albumin using a saliva sample. The result provides an assessment of glycemic control over the preceding 2-3 weeks.

Frequent monitoring of the individuals glycated albumin would provide an accurate assessment of overall effectiveness of glycemic control in the individual and allow earlier therapeutic intervention compared to the glycated hemoglobin test in current use.

The present invention describes a simplified point-of-care assay that utilizes disposable test strips and a reusable measuring instrument.

### BRIEF DESCRIPTION OF THE DRAWINGS

FIG. 1. is an illustration of the disposable test strip containing the reagents and the placement of the components required to measure glycated and non-glycated albumin.

FIG. 2. is an illustration of a fluorescence measuring instrument into which the test strip is inserted. The indicator agent used in the test strip is a fluorescing compound and the amount of fluorescence measured at the glycated albumin band region and at the non-glycated albumin band region is used to calculated the ratio of glycated albumin to total albumin in the sample.

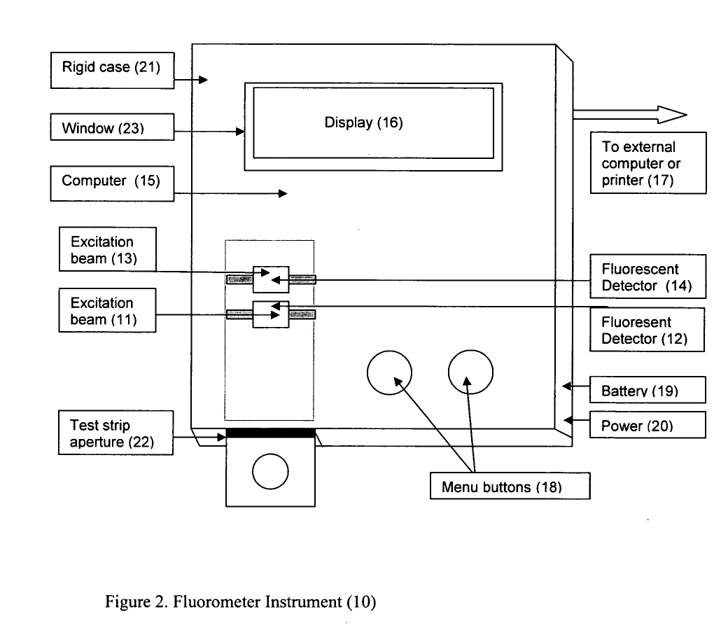

FIG. 3. is an illustration of a spectrophotometer measuring instrument into which the test strip is inserted. The indicator agent used in the test strip is a a colored compound or substance and the amount of color measured at the glycated albumin band region and at the non-glycated albumin band region is used to calculated the ratio of glycated albumin to total albumin in the sample.

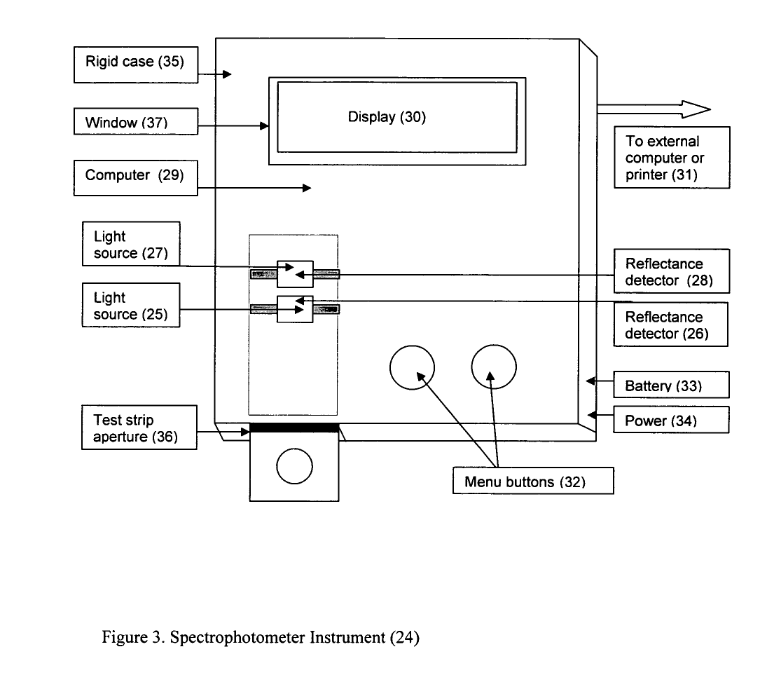

### DESCRIPTION OF THE INVENTION

This invention describes a procedure for measuring the percent of glycated albumin compared to total albumin in the patient's blood and/or saliva. The patient's blood or saliva sample is placed in a test cassette that contains reagents to perform the test. The test cassette is then inserted into a measuring instrument that reads, calculates, stores and reports the result.

Principle: The Rapid Assay for glycated albumin utilizes antibodies to glycated albumin and antibodies to total albumin.

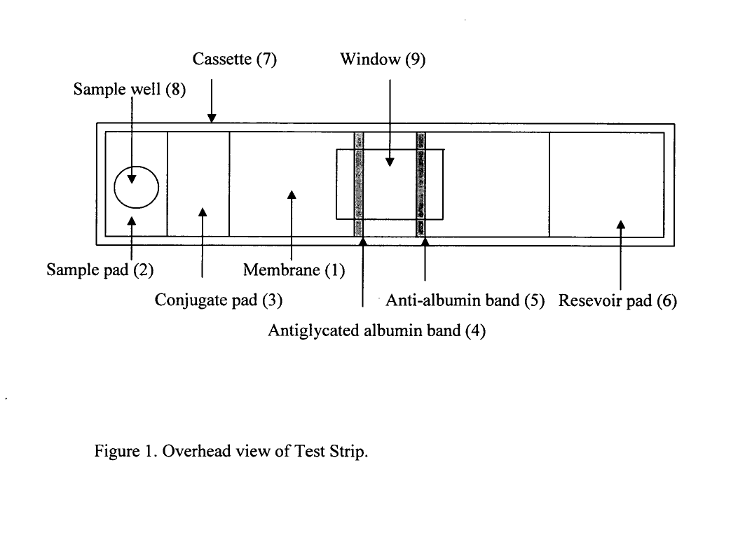

The test strip for measuring glycated albumin is shown in FIGS. 1. It consists of a cellulose nitrate membrane (1) or similar membrane support. There is a sample application pad (2) that serves to remove particulate material and allow the fluid component to flow through. Distal to the sample application pad there is band of anti-albumin antibody labeled with an indicator agent (3). Further along the membrane there is a band of anti-glycated albumin antibody (4) fixed to the membrane; and further along the membrane there is a band of anti-albumin antibody (5) fixed to the membrane; and further along the membrane there is a reservoir pad (6) at the distal end of the membrane. The test strip is enclosed within a rigid cassette (7) containing a sample well (8) and window segments (9) to allow for measurement of the test result using a measuring instrument such as a fluorometer or spectrometer or other biosensor instrumentation.

All the measuring instruments share the same basic design. In the fluorometer (10) there is an exicitation beam of light emitter (11) at the glycated albumin band with its corresponding fluorescence detector (12); and another excitation beam of light emitter (13) at the non-glycated albumin band with its corresponding fluorescence detector (14). The intensity of fluorescence from each band is measured and used to calculate the result. There is an on-board computer (15) that performs the calculations and reports the result, which is displayed on a liquid crystal display (16) or sent to an external computer or printer (17). Commands to the computer are made via a set of keys or menu buttons (18). The instrument is powered by a battery (19) or external power source (20). The external case is made of a rigid material (21) with an aperture (22) for insertion of the test cassette and a window (23) for the LCD.

The spectrometer (24) used for measuring the intensity of color has a light source (25) to illuminate the glycated albumin band and a corresponding detector (26) to measure the color intensity of the glycated albumin band. There is a light source (27) to illuminate the non-glycated albumin band with its corresponding detector (28) to measure the color intensity of the non-glycated albumin band. The color intensity from each band is measured and used to calculate the result. There is an on-board computer (29) that performs the calculations and reports the result, which is displayed on a liquid crystal display (30) or sent to an external computer or printer (31). Commands to the computer are made via a set of keys or menu buttons (32). The instrument is powered by a battery (33) or external power source (34). The external case is made of a rigid material (35) with an aperture (36) for insertion of the test cassette and a window (37) for the LCD.

Other types of measuring instruments may be similarly employed and are within the scope of this invention.

Description of the components and test procedure.

A saliva sample is placed in the sample well and allowed to absorb into the sample application pad. The sample application has a porosity that will filter out particulate material and allow the filtrate to flow through.

The saliva sample then migrates along the membrane and mixes with the labeled anti-albumin antibody reagent. The labeled reagent binds to the albumin present in the sample and the resultant immune complex migrates along the membrane until it contacts the band of fixed anti-glycated albumin antibody. The anti-glycated albumin antibody will bind and fix any immune complex containing glycated albumin and in turn the indicator reagent moiety of the immune complex also becomes fixed. The remaining immune complexes that do not contain glycated albumin are not bound and continue to migrate along the membrane until they contact the band of fixed anti-albumin antibody. The anti-albumin antibody will bind and fix the immune complexes containing albumin and in turn the indicator reagent also is fixed to the membrane.

The intensity of the glycated albumin band and the non-glycated albumin band are measured in a measuring instrument. The measuring instrument used will depend upon the type of indicator that was used to label the albumin. For example, if a fluorescein label is used then the measuring instrument is a fluorometer designed for this purpose; or if an enzyme label is used then the measuring instrument is a spectrometer designed for this purpose, or if colloidal gold or latex particles are used then the measuring instrument may be a reflectance spectrophotometer.

The intensity of the bands are directly proportional to the amount of glycated albumin and non-glycated albumin present in the saliva sample. The intensity of the bands are measured by the measuring instrument that also calculates the result according to a reference standard and a mathematical algorithm based on the formula:

Percentage ratio of glycated albumin compared to total albumin is

$$\frac{A \times 100}{( A + B )}$$

where A is the glycated albumin band and B is the non-glycated albumin band.

The result is expressed as the percent of glycated albumin to total albumin and displayed on the instrument's display screen.

To monitor diabetic control the test is performed on a periodic basis and the results of successive testing are stored in the measuring instrument's memory. The results can be expressed as a numerical display and/or in a graphical format so that trend analysis of glycemic control over time can be performed. The results can also be sent to an external computer and/or printer for further storage and display.

Materials:

The materials for this assay can produced according to standard laboratory methods or purchased commercially. The membrane employed is a cellulose nitrate membrane or similar porous membrane.

The anti-albumin antibodies are prepared in immunized animals such as

The anti-albumin antibodies are prepared in immunized animals such as rabbits, sheep, goats, or other immunized species of animals, or by monoclonal antibody techniques. Either the whole antiserum, or the IgG purified fraction, or the affinity purified antibody to albumin, or the binding fragments (F ab or F ab2) of the antibody, may be employed. The methods for immunization of animals and the preparation and purification of antibody is performed according to standard laboratory procedures and known to those skilled in the art.

The anti-albumin antibody is labeled with fluorescein or an enzyme according to standard laboratory techniques that are familiar to those skilled in the art. For example, to label the antibody with fluorescein the antibody is mixed with fluorescein isothiocyanate and allowed to react. The fluorescein labeled antibody is then separated from free fluorescein using dialysis, gel-filtration or chromatography techniques.

Alternatively, the anti-albumin antibody can be labeled with an enzyme such as horse radish peroxidase using standard laboratory techniques that are familiar to those skilled in the art. For example, to label the antibody with horse radish peroxidase the antibody is mixed with horse radish peroxidase enzyme and glutaldehyde solution. After the reaction the enzyme labeled antibody is separated from free enzyme using dialysis, gel-filtration or chromatography techniques.

Alternatively, the anti-albumin antibody may be used to coat colloidal gold particles or colored latex beads. The colloidal gold particles and latex particles are selected to have a diameter size of either 5 nm or 10 nm or 20 nm or 40 nm or some integral diameter within this size range of 5 nm to 50 nm.

These and other indicator labels for labeling the anti-albumin antibody are known to those skilled in the art and are within the scope of this invention.

The anti-glycated albumin antibodies are prepared in immunized animals such as rabbits, sheep, goats, or other immunized species of animals, or by monoclonal antibody techniques. Either the whole antiserum, or the IgG purified fraction, or the affinity purified antibody to albumin, or the binding fragments (F ab or F ab2) of the antibody, may be employed. The methods for preparing monoclonal antibody and the preparation and purification of antibody is performed according to standard laboratory procedures and known to those skilled in the art.

The anti-glycated antibodies are diluted in a suitable coating buffer and applied as a band across the membrane and become fixed to the membrane upon drying or further treatment. These methods are known to those skilled in the art and are within the scope of this invention.

Alternatively, instead of using anti-glycated antibody it is possible to replace the antibody with chemicals known to bind glycated proteins such as phenyl boronic acids. The phenyl boronic acid is applied as a band to the membrane strip and will become fixed to the membrane upon drying or further treatment. These methods are known to those skilled in the art and are within the scope of this invention.

The anti-albumin antibodies used for binding to the membrane are prepared in immunized animals such as rabbits, sheep, goats, or other immunized species of animals, or by monoclonal antibody techniques. Either the whole antiserum, or the IgG purified fraction, or the affinity purified antibody to albumin, or the binding fragments (F ab or F ab2) of the antibody, may be employed. The methods for immunization of animals and the preparation and purification of antibody is performed according to standard laboratory procedures and known to those skilled in the art.

In the preferred embodiment of this invention monoclonal anti-albumin antibodies are used to be labeled with the indicator reagent and polyclonal antibodies anti-albumin antibodies are used to prepare the fixed band to the membrane.

The general process for preparing rapid immunochromatographic lateral flow assays are employed in this invention. These methods are known to those skilled in the art and do not affect the novelty of this invention which describes a method for assessing glycemic control by measuring the ratio of glycated albumin to total albumin in a saliva sample.

What is claimed is:

1. An immunochromatographic procedure for measuring glycated albumin in a saliva sample using a test strip and a measuring instrument that measures glycated albumin and non-glycated albumin.

2. According to claim 1 the measuring instrument is a reflectance spectrophotometer or fluorometer or other biosensor instrumentation that reads, calculates and displays the result as the percentage of glycated albumin compared to total albumin in the sample.

3. According to claim 1 the procedure to measure glycated albumin consists of a test strip that has either immobilized anti-glycated albumin antibody or immobilized phenyl boronic acid, fixed as a band on a membrane strip; and a second band of anti-albumin antibody immobilized at a further point along the membrane strip.

4. According to claims 1 and 3 the antibody to glycated albumin may be prepared in immunized animals or produced as a monoclonal antibody.

5. According to claim 4 the anti-glycated albumin antibody used may be the complete molecule, or the IgG purified fraction of the antiserum, or the purified antibody, or the binding fragment (F ab or Fab2) of the antibody.

6. According to claims 1 and 3 the antibody to albumin may be prepared in immunized animals or produced as a monoclonal antibody.

7. According to claim 6 the anti-albumin antibody used may be the complete molecule, or the IgG purified fraction of the antiserum, or the purified antibody, or the binding fragment (F ab or Fab2) of the antibody.

8. According to claim 2 the measuring instrument is either a reflectance spectrophotometer or a fluorometer or a biosensor instrument composed of the following elements: a first means of measuring the glycated albumin band and a second means of measuring the non-glycated albumin band; an internal computer chip for measurement and calculation; a liquid crystal display; a external port to transfer data to an external computer and/or printer; a battery and/or an external power source; and a rigid external case with an aperture for inserting the test cassette.

9. According to claims 1-8 the test results obtained from testing the same individual over a period of time are stored in the measuring instrument's computer memory. The stored data can be retrieved on demand and the results expressed in a numerical format or in a graphical format. The results can be displayed on the instrument's display monitor and/or transferred to an external computer or printer.
